(19) United States
(12) Patent Application Publication
(10) Pub. No.: US 2018/0246097 A1
(43) Pub. Date: Aug. 30, 2018

(54) METHODS AND COMPOSITIONS FOR DETECTING MYCOTOXINS

(71) Applicant: ADVATECT DIAGNOSTICS, LLC, Carrollton, TX (US)

(72) Inventor: Dennis G. HOOPER, Lewisville, TX (US)

(21) Appl. No.: 15/760,359

(22) PCT Filed: Sep. 16, 2016
(86) PCT No.: PCT/US16/52189

§ 371 Date: Mar. 15, 2018

Related U.S. Application Data
(60) Provisional application No. 62/220,125, filed on Sep. 17, 2015.

(51) Int. Cl. G01N 33/569

(52) U.S. Cl. CPC G01N 33/56961; G01N 2333/385

(57) ABSTRACT

This invention relates to methods and compositions for detecting, quantifying, or identifying mycotoxins. More particularly, the invention relates to methods and compositions for detecting, quantifying, or identifying a gliotoxin, or a derivative thereof, a mycotoxin of a Penicillium species, or a mycotoxin of a Chaetomium species, in the tissues or body fluid samples of patients.

### CROSS-REFERENCE TO RELATED APPLICATIONS

This application claims priority under 35 U.S.C. § 119(e) to U.S. Provisional Application Ser. No. 62/220,125, filed Sep. 17, 2015, which is incorporated herein by reference in its entirety.

### FIELD OF THE DISCLOSURE

This invention relates to methods and compositions for detecting, quantifying, or identifying mycotoxins. More particularly, the invention relates to methods and compositions for detecting, quantifying, or identifying a gliotoxin, or a derivative thereof, a mycotoxin of a Penicillium species, or a mycotoxin of a Chaetomium species, in the tissues or body fluid samples of a patient.

### BACKGROUND AND SUMMARY

Molds (i.e., toxigenic and other septate molds) are ubiquitous in the environment. Mold is the common name for various types of fungi. Molds are usually found in moist, warm environments. Because molds grow in wet or moist indoor environments, people are exposed to molds or their byproducts through either direct contact, or through the air, if molds or mold byproducts are aerosolized. Exposure to molds can cause a number of adverse effects including allergic reactions, asthma attacks, and infections, particularly in individuals with immune system deficiencies.

Adverse effects from molds may occur when individuals are exposed to large doses of chemicals, known as mycotoxins, which are fungal metabolites (Samson et al., 1985; Burge, 1990; Flannigan et al., 1991). Mycotoxins have toxic effects ranging from severe irritations, such as allergic reactions and asthma, to immuno-suppression and cancer. Most mycotoxins are cytotoxic and exert their effects by interfering with vital cellular processes such as protein, RNA, and DNA synthesis. As a result, mycotoxins may be damaging to the skin, the lungs, the gut, and the like. The combined outcome may increase the susceptibility of the exposed individual to infectious diseases and, possibly, to cancer. Almost all of the studies to date focus on disease induced by mycotoxins ingested in contaminated food (Baxter et al., 1981), but mycotoxins are secondary metabolites of fungal spores and can enter the body through the respiratory tract.

In heavily contaminated environments, neurotoxic symptoms related to airborne mycotoxin exposure have been reported (Croft et al., 1986). Skin is another potential route of exposure to the mycotoxins of several fungi which have caused cases of severe dermatosis (Vennewald and Wollina, 2005). These same molds may cause invasive mold infection among patients with diseases which render the patient immuno-suppressed such as leukemia, lymphoma, and many cancers (Kontoyiannis, D P et al, 2005). The mold infections in such patients are often fatal with a documented fatally rate of 92% (Paterson and Singh, 1999).

A definitive and early diagnosis of a fungal infection is crucial for patient treatment and management. A diagnosis of a fungal infection is often rendered late in the disease process, often even as late as autopsy (Kontoyiannis et al, 2000; Vogeser et al., 1997). The reasons for the late diagnosis of fungal infections include the lack of good clinical specimens, the difficultly in differentiating invasive mold infections from other types of infections, the lack of identification of molds with special stains in pathological specimens (i.e., these assays have a high error rate, a low sensitivity, and low specificity), the lack of an ability to obtain an antibody-based diagnosis in immuno-compromised patients, and the lack of assays to determine the presence of mycotoxins in the tissue or body fluids of those patients.

Thus, reliable, sensitive, specific, and rapid methods for mold detection in patient body fluids and tissues are needed. Applicant's present invention is based on the idea that if mycotoxins can be identified in patient tissue or body fluids, the identification, detection, or quantitation of mycotoxins may serve as a potential diagnostic method 1) to identify patients at risk for developing disease states related to mold infections, or 2) to rapidly determine the cause of diseases related to mold infections so that effective treatment regimens can be developed for patients exposed to molds and experiencing symptoms resulting from mold infection. The methods and compositions described herein overcome the deficiencies in the art by providing reliable, sensitive, and specific diagnostic tests for the presence of fungal toxins in patient tissue and body fluids, particularly for gliotoxins, or derivatives thereof, such as Bis-(methylthio)gliotoxin, mycotoxins of Penicillium species, such as mycophenolic acid, and mycotoxins of Chaetomium species, such as emodins, chrysophanols, chaetoglobosins A, B, C, D, E and F, chetomins, azaphilones, and chaetoviridins.

Several illustrative embodiments of the invention are described in the following enumerated clauses:

1. A method of identifying a gliotoxin, or a derivative thereof, a mycotoxin of a Penicillium species, or a mycotoxin of a Chaetomium species in a patient tissue or a body fluid, the method comprising:
extracting the mycotoxin from the patient tissue or the body fluid;
contacting the mycotoxin with an antibody directed against the mycotoxin; and
identifying the myocotoxin wherein the mycotoxin is a gliotoxin, or a derivative thereof, a mycotoxin of a Penicillium species, or a mycotoxin of a Chaetomium species.

2. The method of clause 1 further comprising quantifying the mycotoxin.

3. The method of clause 1 or 2 wherein the body fluid is selected from the group consisting of urine, nasal secretions, nasal washes, bronchial lavages, bronchial washes, spinal fluid, sputum, gastric secretions, seminal fluid, other reproductive tract secretions, lymph fluid, whole blood, serum, and plasma.

4. The method of any one of clauses 1 to 3 wherein the mycotoxin is a gliotoxin derivative.

5. The method of clause 4 wherein the gliotoxin derivative is Bis-(methylthio)gliotoxin.

6. The method of any one of clauses 1 to 3 wherein the mycotoxin is a mycotoxin of a Penicillium species.

7. The method of clause 6 wherein the mycotoxin is mycophenolic acid.

8. The method of any one of clauses 1 to 3 wherein the mycotoxin is a mycotoxin of a Chaetomium species.

9. The method of clause 8 wherein the mycotoxin is selected from the group consisting of emodins, chrysophanols, chaetoglobosins A, B, C, D, E and F, chetomins, azaphilones, and chaetoviridins.

10. The method of clause 9 wherein the mycotoxin is chaetoglobosin A or B.

11. The method of any one of clauses 1 to 10 wherein the antibody is a polyclonal antibody.

12. The method of any one of clauses 1 to 10 wherein the antibody is a monoclonal antibody.

13. The method of any one of clauses 2 to 12 wherein the sensitivity of the quantitation is at least 0.2 ng/ml.

14. The method of clause 5 wherein there is no other mycotoxin detected.

15. The method of clause 7 wherein there is no other mycotoxin detected.

16. The method of clause 10 wherein there is no other mycotoxin detected.

17. The method of any one of clauses 1 to 16 wherein the mycotoxin is contacted with the antibody using an enzyme-linked immunosorbent assay.

18. The method of any one of clauses 1 to 17 further comprising identifying the mycotoxin using negative and positive control samples.

19. The method of any one of clauses 1 to 18 further comprising using calibration reagents to quantify the mycotoxin.

20. The method of any one of clauses 1 to 19 wherein methanol is used for the extraction.

21. A method of detecting a gliotoxin, or a derivative thereof, a mycotoxin of a Penicillium species, or a mycotoxin of a Chaetomium species in a patient tissue or a body fluid, the method comprising:
extracting the mycotoxin from the patient tissue or the body fluid;
contacting the mycotoxin with an antibody directed against the mycotoxin; and
detecting the myocotoxin wherein the mycotoxin is a gliotoxin, or a derivative thereof, a mycotoxin of a Penicillium species, or a mycotoxin of a Chaetomium species.

22. The method of clause 21 further comprising quantifying the mycotoxin.

23. The method of clause 21 or 22 wherein the body fluid is selected from the group consisting of urine, nasal secretions, nasal washes, bronchial lavages, bronchial washes, spinal fluid, sputum, gastric secretions, seminal fluid, other reproductive tract secretions, lymph fluid, whole blood, serum, and plasma.

24. The method of any one of clauses 21 to 23 wherein the mycotoxin is a gliotoxin derivative.

25. The method of clause 24 wherein the gliotoxin derivative is Bis-(methylthio)gliotoxin.

26. The method of any one of clauses 21 to 23 wherein the mycotoxin is a mycotoxin of a Penicillium species.

27. The method of clause 26 wherein the mycotoxin is mycophenolic acid.

28. The method of any one of clauses 21 to 23 wherein the mycotoxin is a mycotoxin of a Chaetomium species.

29. The method of clause 28 wherein the mycotoxin is selected from the group consisting of emodins, chrysophanols, chaetoglobosins A, B, C, D, E and F, chetomins, azaphilones, and chaetoviridins.

30. The method of clause 29 wherein the mycotoxin is chaetoglobosin A or B.

31. The method of any one of clauses 21 to 30 wherein the antibody is a polyclonal antibody.

32. The method of any one of clauses 21 to 30 wherein the antibody is a monoclonal antibody.

33. The method of any one of clauses 22 to 32 wherein the sensitivity of the quantitation is at least 0.2 ng/ml.

34. The method of clause 25 wherein there is no other mycotoxin detected.

35. The method of clause 27 wherein there is no other mycotoxin detected.

36. The method of clause 30 wherein there is no other mycotoxin detected.

37. The method of any one of clauses 21 to 36 wherein the mycotoxin is contacted with the antibody using an enzyme-linked immunosorbent assay.

38. The method of any one of clauses 21 to 37 further comprising identifying the mycotoxin using negative and positive control samples.

39. The method of any one of clauses 21 to 38 further comprising using calibration reagents to quantify the mycotoxin.

40. The method of any one of clauses 21 to 39 wherein methanol is used for the extraction.

41. A method of quantifying a gliotoxin, or a derivative thereof, a mycotoxin of a Penicillium species, or a mycotoxin of a Chaetomium species in a patient tissue or a body fluid, the method comprising:
extracting the mycotoxin from the patient tissue or the body fluid;
contacting the mycotoxin with an antibody directed against the mycotoxin; and
quantifying the myocotoxin wherein the mycotoxin is a a gliotoxin, or a derivative thereof, a mycotoxin of a Penicillium species, or a mycotoxin of a Chaetomium species.

42. The method of clause 41 further comprising quantifying the mycotoxin.

43. The method of clause 41 or 42 wherein the body fluid is selected from the group consisting of urine, nasal secretions, nasal washes, bronchial lavages, bronchial washes, spinal fluid, sputum, gastric secretions, seminal fluid, other reproductive tract secretions, lymph fluid, whole blood, serum, and plasma.

44. The method of any one of clauses 41 to 43 wherein the mycotoxin is a gliotoxin derivative.

45. The method of clause 44 wherein the gliotoxin derivative is Bis-(methylthio)gliotoxin.

46. The method of any one of clauses 41 to 43 wherein the mycotoxin is a mycotoxin of a Penicillium species.

47. The method of clause 46 wherein the mycotoxin is mycophenolic acid.

48. The method of any one of clauses 41 to 43 wherein the mycotoxin is a mycotoxin of a Chaetomium species.

49. The method of clause 48 wherein the mycotoxin is selected from the group consisting of emodins, chrysophanols, chaetoglobosins A, B, C, D, E and F, chetomins, azaphilones, and chaetoviridins.

50. The method of clause 49 wherein the mycotoxin is chaetoglobosin A or B.

51. The method of any one of clauses 41 to 50 wherein the antibody is a polyclonal antibody.

52. The method of any one of clauses 41 to 50 wherein the antibody is a monoclonal antibody.

53. The method of any one of clauses 42 to 52 wherein the sensitivity of the quantitation is at least 0.2 ng/ml.

54. The method of clause 45 wherein there is no other mycotoxin detected.

55. The method of clause 47 wherein there is no other mycotoxin detected.

56. The method of clause 50 wherein there is no other mycotoxin detected.

57. The method of any one of clauses 41 to 56 wherein the mycotoxin is contacted with the antibody using an enzyme-linked immunosorbent assay.

58. The method of any one of clauses 41 to 57 further comprising identifying the mycotoxin using negative and positive control samples.

59. The method of any one of clauses 41 to 58 further comprising using calibration reagents to quantify the mycotoxin.

60. The method of any one of clauses 41 to 59 wherein methanol is used for the extraction.

61. A method of determining if a patient is at risk for or has developed a fungal infection wherein the fungal infection produces a gliotoxin, or a derivative thereof, a mycotoxin of a Penicillium species, or a mycotoxin of a Chaetomium species, the method comprising:
extracting the mycotoxin from a tissue or a body fluid of the patient;
contacting the mycotoxin with an antibody directed against the mycotoxin;
identifying the mycotoxin; and
determining if the patient is at risk for or has developed the fungal infection wherein the fungal infection produces a gliotoxin, or a derivative thereof, a mycotoxin of a Penicillium species, or a mycotoxin of a Chaetomium species.

62. The method of clause 61 further comprising quantifying the mycotoxin.

63. The method of clause 61 or 62 wherein the body fluid is selected from the group consisting of urine, nasal secretions, nasal washes, bronchial lavages, bronchial washes, spinal fluid, sputum, gastric secretions, seminal fluid, other reproductive tract secretions, lymph fluid, whole blood, serum, and plasma.

64. The method of any one of clauses 61 to 63 wherein the mycotoxin is a gliotoxin derivative.

65. The method of clause 64 wherein the gliotoxin is Bis-(methylthio)gliotoxin.

66. The method of any one of clauses 61 to 63 wherein the mycotoxin is a mycotoxin of a Penicillium species.

67. The method of clause 66 wherein the mycotoxin is mycophenolic acid.

68. The method of any one of clauses 61 to 63 wherein the mycotoxin is a mycotoxin of a Chaetomium species.

69. The method of clause 68 wherein the mycotoxin is selected from the group consisting of emodins, chrysophanols, chaetoglobosins A, B, C, D, E and F, chetomins, azaphilones, and chaetoviridins.

70. The method of clause 69 wherein the mycotoxin is a chaetoglobosin A or B.

71. The method of any one of clauses 61 to 70 wherein the antibody is a polyclonal antibody.

72. The method of any one of clauses 61 to 70 wherein the antibody is a monoclonal antibody.

73. The method of any one of clauses 62 to 72 wherein the sensitivity of the quantitation is at least 0.2 ng/ml.

74. The method of clause 65 wherein there is no other mycotoxin detected.

75. The method of clause 67 wherein there is no other mycotoxin detected.

76. The method of clause 70 wherein there is no other mycotoxin detected.

77. The method of any one of clauses 61 to 76 wherein the mycotoxin is contacted with the antibody using an enzyme-linked immunosorbent assay.

78. The method of any one of clauses 61 to 77 further comprising identifying the mycotoxin using negative and positive control samples.

79. The method of any one of clauses 61 to 78 further comprising using calibration reagents to quantify the mycotoxin.

80. The method of any one of clauses 61 to 79 wherein methanol is used for the extraction.

81. The method of any one of clauses 61 to 80 further comprising developing an effective treatment regimen for the patient.

82. The method of clause 81 wherein the treatment regimen comprises administering an antifungal drug to the patient.

83. A kit comprising components for the extraction of a gliotoxin, or a derivative thereof, a mycotoxin of a Penicillium species, or a mycotoxin of a Chaetomium species from a body fluid or a tissue of a patient.

84. The kit of clause 83 further comprising an antibody directed against a gliotoxin, or a derivative thereof, a mycotoxin of a Penicillium species, or a mycotoxin of a Chaetomium species.

85. The kit of clause 83 or 84 wherein the body fluid is selected from the group consisting of urine, nasal secretions, nasal washes, bronchial lavages, bronchial washes, spinal fluid, sputum, gastric secretions, seminal fluid, other reproductive tract secretions, lymph fluid, whole blood, serum, and plasma.

86. The kit of any one of clauses 83 to 85 wherein the mycotoxin is a gliotoxin derivative.

87. The kit of clause 86 wherein the gliotoxin derivative is Bis-(methylthio)gliotoxin.

88. The kit of any one of clauses 83 to 85 wherein the mycotoxin is a mycotoxin of a Penicillium species.

89. The kit of clause 88 wherein the mycotoxin is mycophenolic acid.

90. The kit of any one of clauses 83 to 85 wherein the mycotoxin is a mycotoxin of a Chaetomium species.

91. The kit of clause 90 wherein the mycotoxin is selected from the group consisting of emodins, chrysophanols, chaetoglobosins A, B, C, D, E and F, chetomins, azaphilones, and chaetoviridins.

92. The kit of clause 91 wherein the mycotoxin is chaetoglobosin A or B.

93. The kit of any one of clauses 84 to 92 wherein the antibody is a polyclonal antibody.

94. The kit of any one of clauses 84 to 92 wherein the antibody is a monoclonal antibody.

95. The kit of any one of clauses 83 to 94 wherein the kit is capable of quantitating the mycotoxin and the sensitivity of the quantitation is at least 0.2 ng/ml.

96. The kit of any one of clauses 83 to 95 further comprising negative and positive control samples.

97. The kit of any one of clauses 83 to 96 further comprising calibration reagents.

98. The kit of any one of clauses 83 to 97 further comprising methanol for extraction.

99. The method or kit of any of the preceding clauses wherein the gliotoxin derivative has the formula

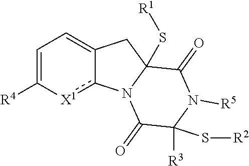

wherein
R¹ and R² are each independently H or C₁-C₄ alkyl, or R¹ and R² are taken together to form a bond;
R³ is selected from the group consisting of H, C₁-C₆ alkyl, C₂-C₆ alkenyl, —(CH₂)ₙOR⁷, —C(O)R⁷, —C(O)OR⁷ and —C(O)NR⁷R⁷;
R⁴ is selected from the group consisting of H, —OR⁸, and —OC(O)R⁸;
R⁵ is selected from the group consisting of H, C₁-C₆ alkyl, C₂-C₆ alkenyl, —C(O)R⁹, —C(O)OR⁹ and —C(O)NR⁹R^{9′};
X¹ is —C(R⁶)═ or —C(R⁶) (R^{6′})—;
R⁶ and R^{6′} are each independently selected from the group consisting of H, —OR¹⁰, and —OC(O)R¹⁰;
R⁷, R^{7′}, R⁸, R⁹, R^{9′}, and R¹⁰ are each independently selected from the group consisting of H, C₁-C₆ alkyl, C₂-C₆ alkenyl, —C(O)R¹¹, —C(O)OR¹¹ and —C(O)NR¹¹R^{11′};
R¹¹ and R^{11′} are each independently H or C₁-C₆ alkyl; and
n is an integer from 1 to 4; provided that the gliotoxin is not

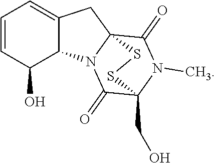 [SMILES: CN1C(=O)[C@]23CC4=CC=C[C@H](O)[C@H]4N2C(=O)[C@@]1(CO)SS3]

100. The method or kit of clause 99 wherein R¹ and R² are methyl.

101. The method or kit of any one of clauses 99 to 100 wherein R³ is —CH₂OR⁷.

102. The method or kit of any one of clauses 99 to 101 wherein R⁷ is H.

103. The method or kit of any one of clauses 99 to 102 wherein R⁴ is H.

104. The method or kit of any one of clauses 99 to 103 wherein R⁵ is methyl.

105. The method or kit of any one of clauses 99 to 104 wherein X¹ is —C(R⁶)

106. The method or kit of any one of clauses 99 to 105 wherein R⁶ is —OR¹⁰.

107. The method or kit of any one of clauses 99 to 106 wherein R^{6′} is H.

108. The method or kit of any one of clauses 99 to 107 wherein R¹⁰ is H.

109. The method or kit of any one of clauses 99 to 108 wherein the gliotoxin is of the formula

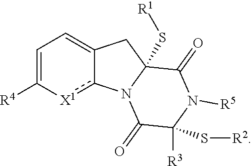

110. The method or kit of any one of clauses 99 to 108 wherein the gliotoxin is of the formula

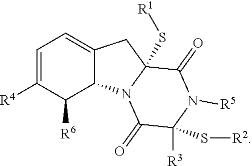

111. The method or kit of any one of clauses 99 to 108 wherein the gliotoxin is of the formula

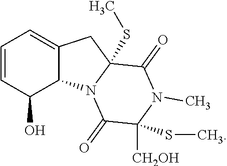 [SMILES: CS[C@]1(CO)C(=O)N2[C@H]3C(=CC=C[C@@H]3O)C[C@@]2(SC)C(=O)N1C]

112. The method of any of the preceding clauses wherein fungal DNA identification is performed in combination with detection, identification, or quantitation of the mycotoxin.

### DETAILED DESCRIPTION OF THE DRAWINGS

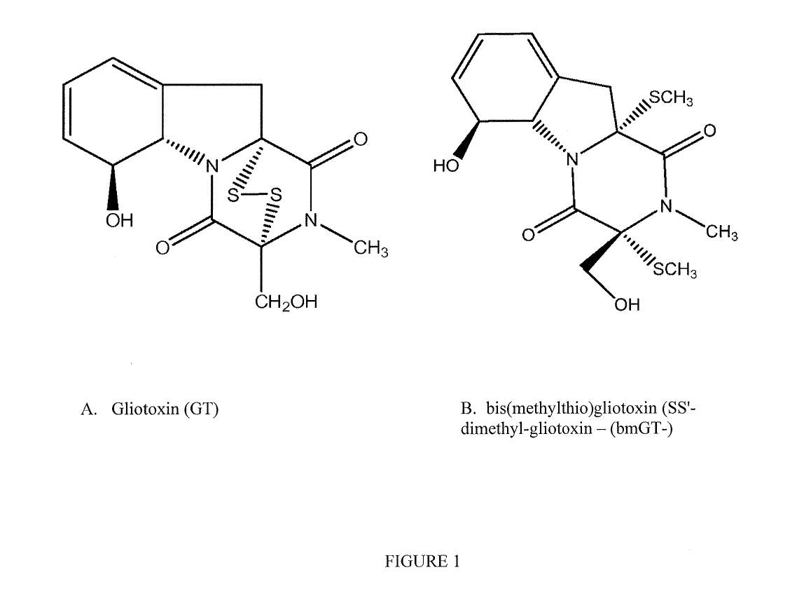

FIG. 1. shows the structures of gliotoxin and Bis-gliotoxin: Panel A shows Gliotoxin (GT) and Panel B shows bis(methylthio)gliotoxin (SS′-dimethyl-gliotoxin—(bmGT-).

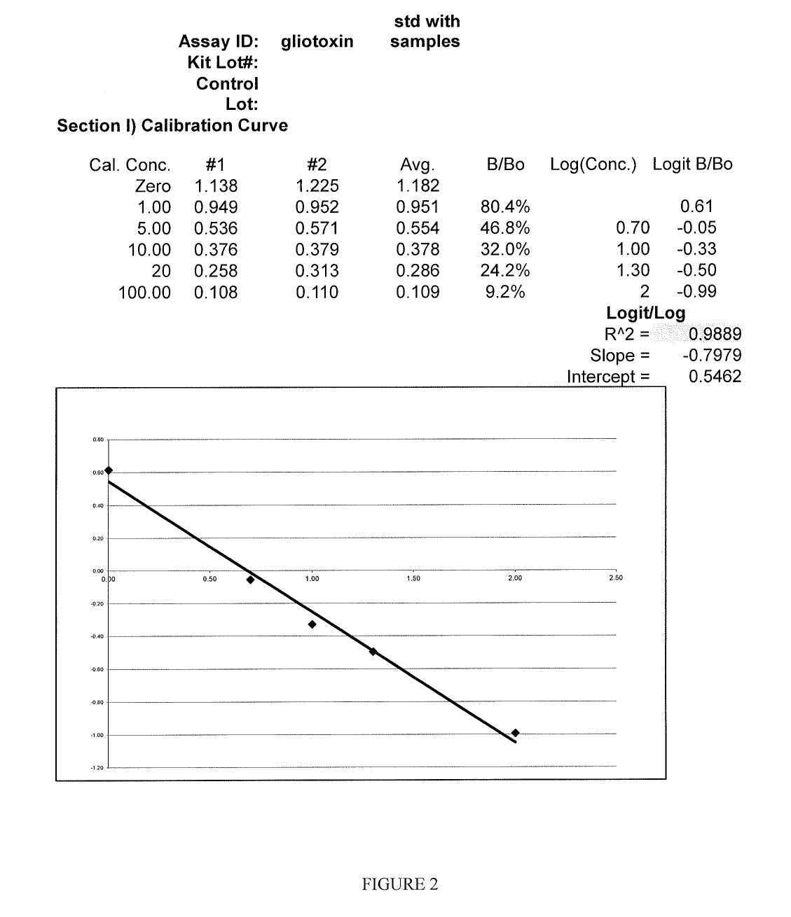

FIG. 2. shows test samples and calibration curve using in-house standard calibrators (gliotoxin).

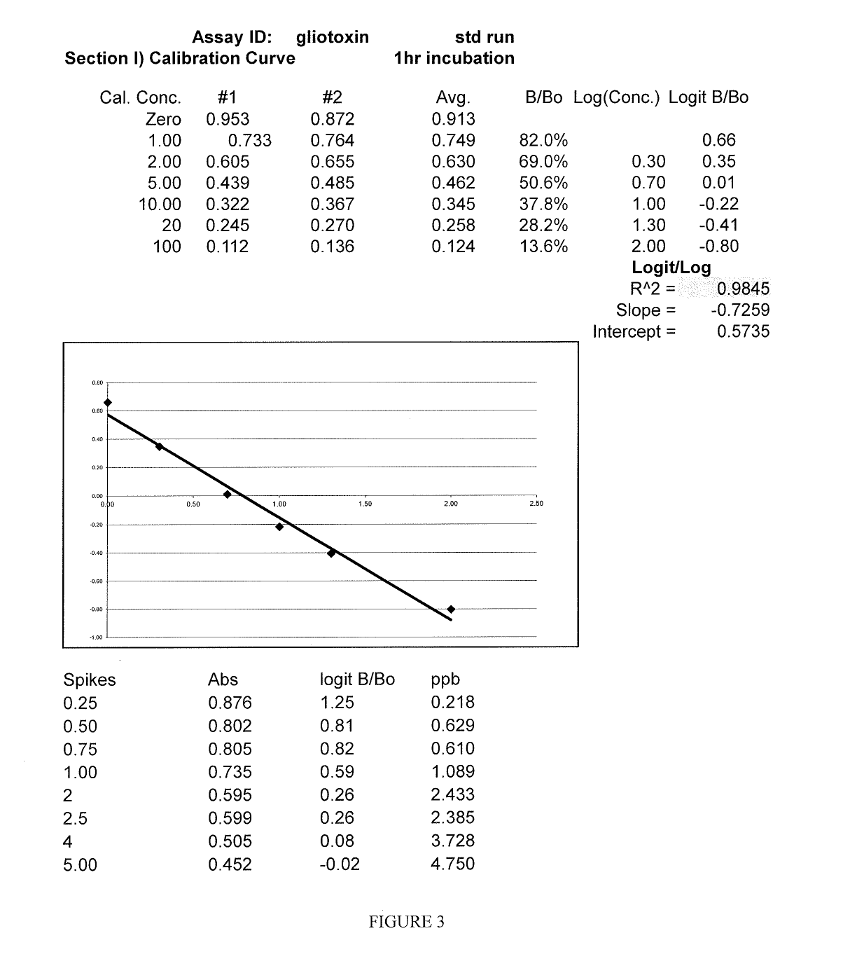

FIG. 3. shows the reproducibility of test samples and calibration curve using standard calibrators (gliotoxin). Standard run with 1 hour incubation.

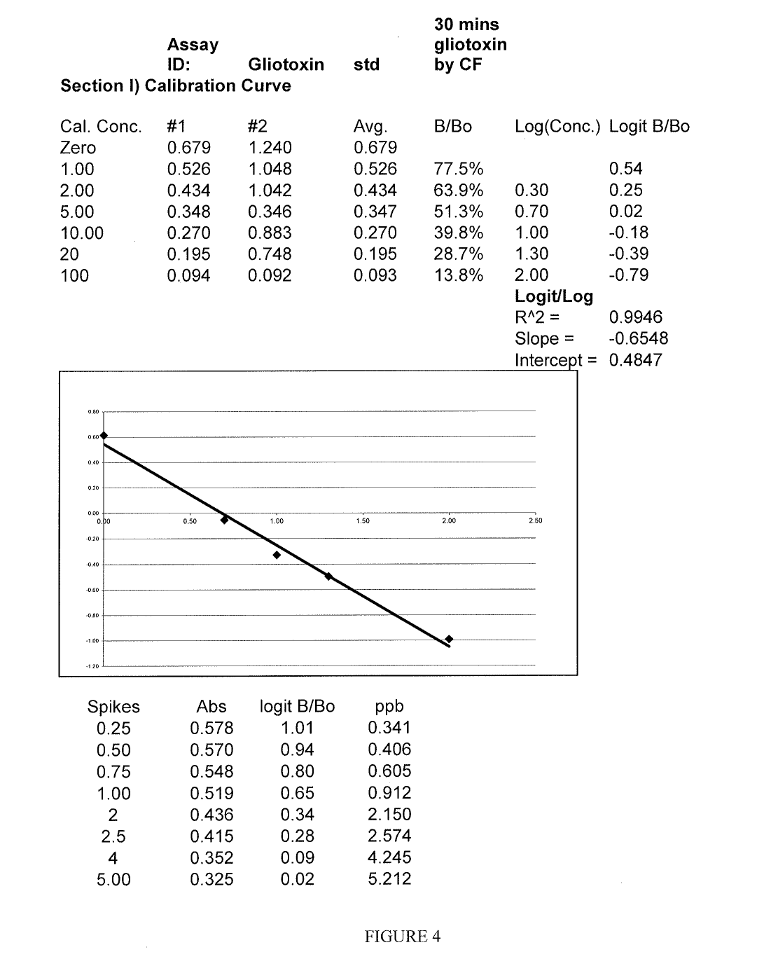

FIG. 4. shows test samples and calibration curve using standard calibrators (gliotoxin). Short run with 30 minute incubation.

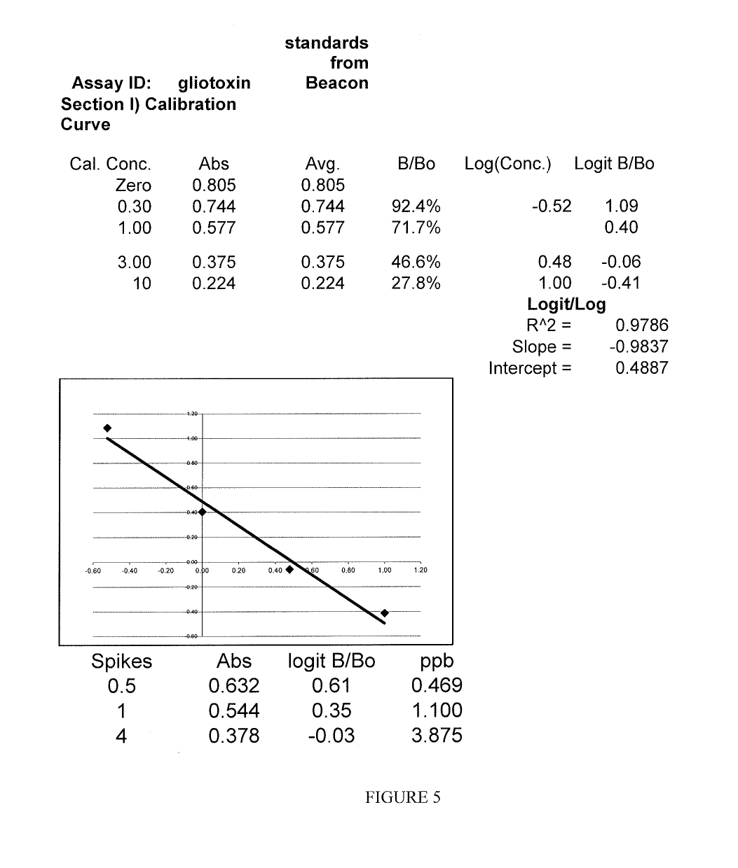

FIG. 5. shows test samples and calibration curve using Beacon Calibrators (gliotoxin).

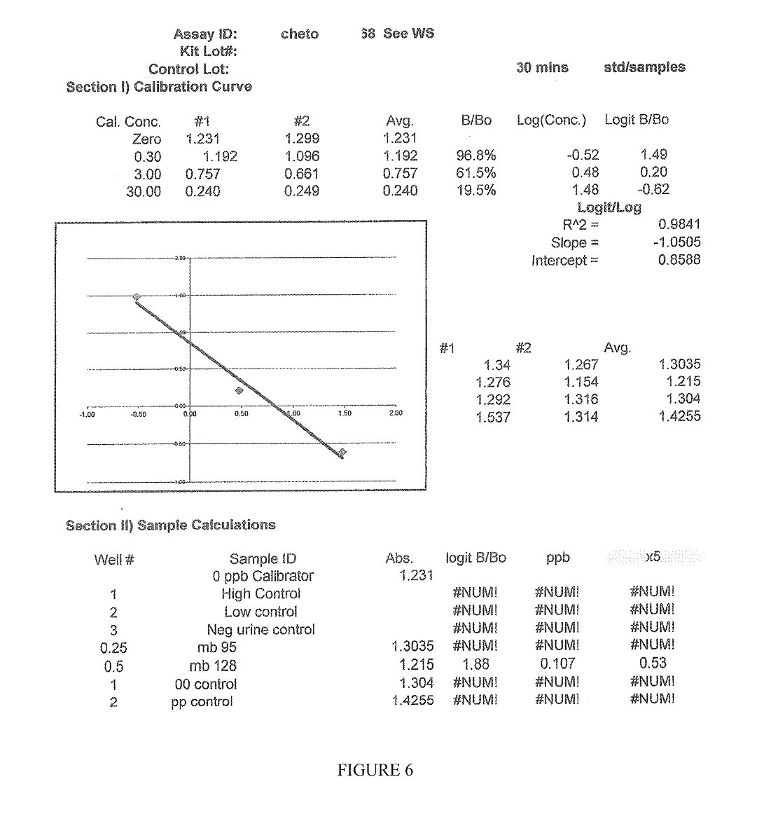

FIG. 6. shows negative controls and calibration curve using standard calibrators (Chaetoglobosin A).

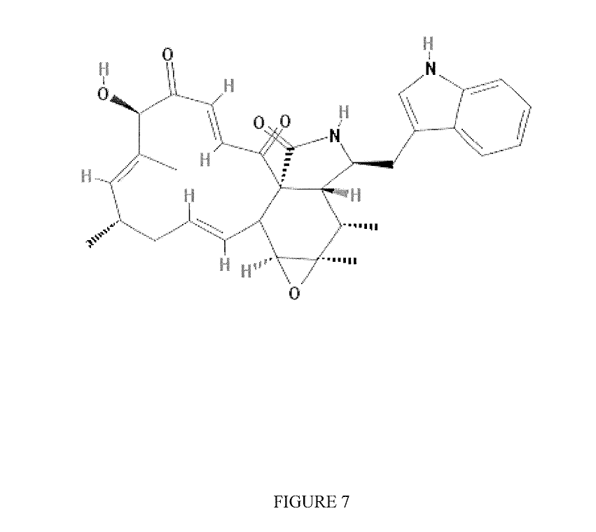

FIG. 7. shows the structure of Chaetoglobosum.

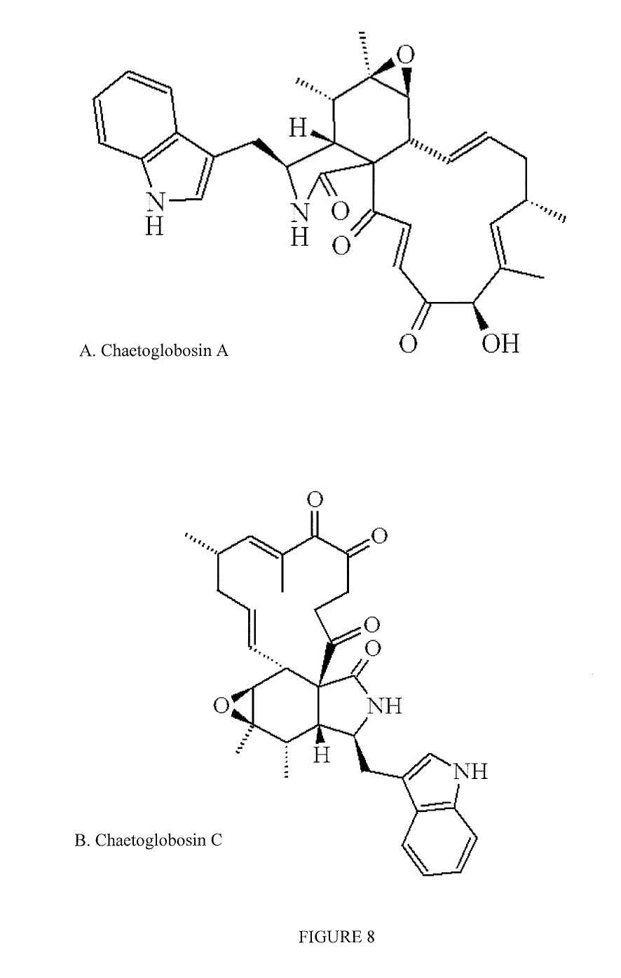

FIG. 8. shows the structures for Chaetoglobosin A (Panel A) and Chaetoglobosin C (Panel B).

### DETAILED DESCRIPTION OF THE ILLUSTRATIVE EMBODIMENTS

Any of the embodiments described in this Detailed Description section can apply to any of the embodiments described in the preceding enumerated clauses, or combinations thereof. In one embodiment, the present invention relates to methods and compositions for identifying, detecting, or quantitating molds (i.e., fungi) in patient tissue and body fluids. In one embodiment, the methods and compositions for detecting, quantifying, or identifying mycotoxins are for detecting, quantifying, or identifying a gliotoxin, or a derivative thereof, a mycotoxin of a Penicillium species, or a mycotoxin of a Chaetomium species, in the tissues or body fluid samples of patients.

In various embodiments, the mycotoxin can be a gliotoxin derivative, such as Bis-(methylthio)gliotoxin, a mycotoxin of a Penicillium species, such as mycophenolic acid, or a derivative thereof, or a mycotoxin of a Chaetomium species, such as chaetoglobosin A or B. In another embodiment, the mycotoxin can be Bis-(methylthio)gliotoxin. In yet another embodiment, the mycotoxin can be mycophenolic acid, or a derivative thereof. In another embodiment, the mycotoxin can be mycophenolic acid. In still another embodiment, the mycotoxin can be chaetoglobosin A or B.

In one aspect, the methods and compositions for detection, identification, and quantification of mycotoxins can also be very specific and sensitive. In exemplary embodiments, the methods and compositions can quantitate mycotoxins with a sensitivity of at least 0.0001ng/ml, at least 0.0003 ng/ml, at least 0.001ng/ml, at least 0.003 ng/ml, at least 0.01 ng/ml, at least 0.02 ng/ml, at least 0.025 ng/ml, at least 0.03 ng/ml, at least 0.04 ng/ml, at least 0.05 ng/ml, at least 0.06 ng/ml, at least 0.07 ng/ml, at least 0.08 ng/ml, at least 0.09 ng/ml, at least 0.1 ng/ml, at least 0.2 ng/ml, at least 0.25 ng/ml, at least 0.3 ng/ml, at least 0.4 ng/ml, at least 0.5 ng/ml, at least 0.6 ng/ml, at least 0.7 ng/ml, at least 0.8 ng/ml, at 0.9 ng/ml, at least 1 ng/ml, at least 2 ng/ml, at least 2.5 ng/ml, at least 3 ng/ml, or at least 0.2, 0.25 or 0.3 ng/dl. In one illustrative aspect, the methods and compositions utilize antibody-based identification of mycotoxins.

In illustrative embodiments, Enzyme Linked Immunosorbant Assay (ELISA), or affinity chromatography can be used to detect the mycotoxins described herein. Illustratively, the mycotoxins can be a gliotoxin, or a derivative thereof, a mycotoxin of a Penicillium species, or a mycotoxin of a Chaetomium species. In another embodiment, the mycotoxin can be a gliotoxin derivative, and the gliotoxin derivative can be Bis-(methylthio)gliotoxin. In another exemplary embodiment, the mycotoxin can be a mycotoxin of a Penicillium species, such as Penicillium brevicompactum, and the mycotoxin can be mycophenolic acid. In yet another embodiment, the mycotoxin can be a mycotoxin of a Chaetomium species, such as Chaetomium globosum, and the mycotoxin of a Chaetomium species can be selected from the group consisting of emodins, chrysophanols, chaetoglobosins A, B, C, D, E and F, chetomins, azaphilones, and chaetoviridins. In another embodiment, the mycotoxin can be chaetoglobosin A or B. Illustrative of antibodies that can be used in the methods described herein are antibodies obtained from Enzo Life Sciences, Inc. (Farmingdale, N.Y.).

In various illustrative embodiments, body fluids that can be tested for the presence of mycotoxins, include, but are not limited to, urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, such as seminal fluid, lymph fluid, and whole blood, serum, or plasma. In some embodiments, these samples can be prepared for testing as described herein or in U.S. Application Publication Number 2008/0014582, incorporated herein by reference. In various embodiments, tissue samples can include tissue biopsies of hospital patients or out-patients and autopsy specimens. As used herein, the term “tissue” includes, but is not limited to, biopsies, autopsy specimens, cell extracts, tissue sections, aspirates, tissue swabs, and fine needle aspirates.

As used herein, the word “patient” means a human or an animal, such as a domestic animal (e.g., a dog or a cat). Accordingly, the methods and compositions disclosed herein can be used for both human clinical medicine and veterinary applications. Thus, in various embodiments, the patient afflicted with a fungal infection can be a human, or in the case of veterinary applications, can be a laboratory, agricultural, domestic or wild animal. In one embodiment, the methods and compositions described herein can be applied to patients including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, chickens, and wild animals in captivity such as bears, pandas, lions, tigers, leopards, elephants, zebras, giraffes, gorillas, dolphins, and whales.

In several embodiments, the methods and compositions described herein can be used to detect, identify, or quantitate microbial toxins (e.g., mycotoxins), such as gliotoxins, or derivatives thereof, in microbes selected from the group consisting of Aspergillus species, Tricoderma species, Penicillium species, Gliocladium species, Thermoascus species, Candida species, and Chaetomium species.

In some embodiments of this method embodiment, the microbe can be selected from the group consisting of A. flavus, A. fumigatus, A. terreus, A. niger, A. versicolor, A. nidulans, A. ochraceus, A. paraciticus, A. sydowii, A. ustus, P. aurantiogriseum, P. citrinum, P. corylophilum, P. crustosum, P. expansum, P. fellutanum, P. roquefortii, and P. simplicissimum, P. brevicompactum, P. chrysogenum, C. globosum, Candida albicans, Candida glabrata, Candida krusei, and Candida tropicalis.

Illustratively, patient (e.g., human or animal) tissue can be received in 1) a 10% formalin fluid or 2) in a paraffin block in which the tissue has been fixed in formalin, such as 10% formalin. In one embodiment for mycotoxin detection, identification, or quantitation, the tissue can then be processed by various dehydration steps and finally embedded in paraffin. In this embodiment, the tissue can then be cut in 3 to 5 micron samples. In an illustrative embodiment, approximately 25 to 35 mg of tissue can then be processed as described in Examples 2 to 3 for mycotoxin extraction, using, for example, methanol for extraction. Illustratively, body fluids can be prepared as described in Examples 1 and 3 or by other methods known in the art. Illustratively, any antigen associated with a fungus or with a mycotoxin can be detected.

In some embodiments, the methods and compositions for detection, identification, or quantification of mycotoxins can be very specific and sensitive. In other embodiments, there may be no cross-over reactions or cross-over detection of mycotoxins between groups, such as individual mycotoxins or classes of mycotoxins from a specific fungal species. In illustrative embodiments, Enzyme-Linked Immunosorbant Assay (ELISA), affinity chromatography, or a Luminex®-based assay can be used to detect, identify, or quantitate mycotoxins produced by toxic molds. Illustratively, the mycotoxins can be a gliotoxin, or a derivative thereof, a mycotoxin of a Penicillium species, or a mycotoxin of a Chaetomium species as described herein.

Another exemplary detection method for multiple mycotoxins in patient samples that have been exposed to fungi that are, for example, Aspergillus species, Tricoderma species, Penicillium species, Gliocladium species, Thermoascus species, Candida species, and Chaetomium species, is the Luminex® format (Luminex, Austin, Tex.). In one aspect of the invention, the Luminex® assay utilizes microspheres (beads) that are dyed with fluorochromes and that are coupled to antigens to detect antibodies, in patient body fluids or tissues, to mycotoxins, mycotoxin antigens, or other fungal antigens. In another embodiment, the microspheres are coupled to antibodies to detect, in patient body fluids or tissues, mycotoxins, mycotoxin antigens, or other fungal antigens. In this illustrative embodiment, the antibodies coupled to the microspheres can be polyclonal or monoclonal antibodies, but monoclonal antibodies are typically used. In another illustrative embodiment, the beads can be coupled to DNA probes to detect DNA specific to fungal species, as described below. In another embodiment, any detection, identification, or quantitation method described in in U.S. Application Publication Number 2008/0014582, incorporated herein by reference, can be used.

In the embodiments where mycotoxins are detected, identified, or quantitated, control samples of the body fluid or tissue to be analyzed can be obtained from patients with no documented history of exposure to molds or mycotoxins. For example, negative control samples can be obtained from autopsy specimens where the patient had no exposure to mycotoxins or molds (e.g., victims of motor vehicle accidents, coronary artery disease, or myocardial infarction). For positive controls, for example, samples of negative tissue and/or body fluids can be spiked with known positive amounts of the mycotoxins described herein or spores prior to evaluation to generate a calibration curve.

In another embodiment, a method of determining if a patient is at risk for or has developed a fungal infection wherein the fungal infection produces a gliotoxin, or a derivative thereof, a mycotoxin of a Penicillium species, or a mycotoxin of a Chaetomium species, is provided. The method comprises extracting the mycotoxin from a tissue or a body fluid of the patient, contacting the mycotoxin with an antibody directed against the mycotoxin, identifying the mycotoxin, and determining if the patient is at risk for or has developed the fungal infection wherein the fungal infection produces a gliotoxin, or a derivative thereof, a mycotoxin of a Penicillium species, or a mycotoxin of a Chaetomium species. In another embodiment, the method further comprises quantifying the mycotoxin.

In this method embodiment, the method can further comprise developing an effective treatment regimen for the patient. In one aspect, the treatment regimen can involve administering to the patient an antifungal drug, such as amphotericin B, caspofungin, or voriconazole.

In any embodiment involving “determining if the patient has developed a fungal infection,” this phrase can mean “diagnosing the patient with a fungal infection.” In various embodiments, patients in need of diagnosis of a fungal infection can include cancer patients, post-operative patients, transplant patients, patients undergoing chemotherapy, immunosuppressed patients, and the like. In some aspects, these patients may experience symptoms of fungal infections including sinusitis, allergic reactions, headaches, and skin rashes. Illustratively, patients in need of diagnosis may include humans or animals.

In various embodiments of this method embodiment, the mycotoxin can be a gliotoxin derivative, such as Bis-(methylthio)gliotoxin, a mycotoxin of a Penicillium species, such as mycophenolic acid, or a mycotoxin of a Chaetomium species, such as chaetoglobosin A or B. In another embodiment, the mycotoxin can be Bis-(methylthio)gliotoxin. In yet another embodiment, the mycotoxin can be mycophenolic acid, or a derivative thereof. In another aspect, the mycotoxin can be mycophenolic acid. In still another embodiment, the mycotoxin can be chaetoglobosin A or B.

In one aspect of this method embodiment, the method can quantitate mycotoxins with a sensitivity of at least 0.0001ng/ml, at least 0.0003 ng/ml, at least 0.001ng/ml, at least 0.003 ng/ml, at least 0.01 ng/ml, at least 0.02 ng/ml, at least 0.025 ng/ml, at least 0.03 ng/ml, at least 0.04 ng/ml, at least 0.05 ng/ml, at least 0.06 ng/ml, at least 0.07 ng/ml, at least 0.08 ng/ml, at least 0.09 ng/ml, at least 0.1 ng/ml, at least 0.2 ng/ml, at least 0.25 ng/ml, at least 0.3 ng/ml, at least 0.4 ng/ml, at least 0.5 ng/ml, at least 0.6 ng/ml, at least 0.7 ng/ml, at least 0.8 ng/ml, at 0.9 ng/ml, at least 1 ng/ml, at least 2 ng/ml, at least 2.5 ng/ml, at least 3 ng/ml, or at least 0.2, 0.25 or 0.3 ng/dl. In one illustrative aspect, the methods and compositions utilize antibody-based identification of mycotoxins.

In illustrative embodiments of this method embodiment, Enzyme Linked Immunosorbant Assay (ELISA), or affinity chromatography can be used to detect the mycotoxins described herein. Illustratively, the mycotoxins can be a gliotoxin, or a derivative thereof, a mycotoxin of a Penicillium species, or a mycotoxin of a Chaetomium species, in the tissues or body fluid samples of patients. In another embodiment, the mycotoxin can be a gliotoxin derivative, and the gliotoxin derivative can be Bis-(methylthio)gliotoxin. In another exemplary embodiment, the mycotoxin can be a mycotoxin of a Penicillium species, such as Penicillium brevicompactum, and the mycotoxin can be mycophenolic acid. In yet another embodiment, the mycotoxin can be a mycotoxin of a Chaetomium species, such as Chaetomium globosum, and the mycotoxin of a Chaetomium species can be selected from the group consisting of emodins, chrysophanols, chaetoglobosins A, B, C, D, E and F, chetomins, azaphilones, and chaetoviridins. Illustrative of antibodies that can be used are antibodies obtained from Enzo Life Sciences, Inc. (Farmingdale, N.Y.). In another embodiment, the Luminex® assay described above can be used.

In various illustrative embodiments of this method embodiment, body fluids that can be tested for the presence of mycotoxins, include, but are not limited to, urine, nasal secretions, nasal washes, inner ear fluids, bronchial lavages, bronchial washes, alveolar lavages, spinal fluid, bone marrow aspirates, sputum, pleural fluids, synovial fluids, pericardial fluids, peritoneal fluids, saliva, tears, gastric secretions, stool, reproductive tract secretions, such as seminal fluid, lymph fluid, and whole blood, serum, or plasma. In some embodiments, these samples can be prepared for testing as described herein or in U.S. Application Publication Number 2008/0014582, incorporated herein by reference. In various embodiments, tissue samples can include tissue biopsies of hospital patients or out-patients.

As used in this method embodiment and herein, the word “patient” means a human or an animal, such as a domestic animal (e.g., a dog or a cat). Accordingly, this method embodiment can be used for both human clinical medicine and veterinary applications. Thus, in various embodiments, the patient afflicted with a fungal infection can be a human, or in the case of veterinary applications, can be a laboratory, agricultural, domestic or wild animal. In one embodiment, the method can be applied to patients including, but not limited to, humans, laboratory animals such rodents (e.g., mice, rats, hamsters, etc.), rabbits, monkeys, chimpanzees, domestic animals such as dogs, cats, and rabbits, agricultural animals such as cows, horses, pigs, sheep, goats, chickens, and wild animals in captivity such as bears, pandas, lions, tigers, leopards, elephants, zebras, giraffes, gorillas, dolphins, and whales.

In several embodiments of this method embodiment, the method can be used to detect, identify, or quantitate microbial toxins (e.g., mycotoxins), such as gliotoxins, or derivatives thereof, in microbes selected from the group consisting of Aspergillus species, Tricoderma species, Penicillium species, Gliocladium species, Thermoascus species, Candida species, and Chaetomium species.

In some embodiments of this method embodiment, the microbe can be selected from the group consisting of A. flavus, A. fumigatus, A. terreus, A. niger, A. versicolor, A. nidulans, A. ochraceus, A. paraciticus, A. sydowii, A. ustus, P. aurantiogriseum, P. citrinum, P. corylophilum, P. crustosum, P. expansum, P. fellutanum, P. roquefortii, and P. simplicissimum, P. brevicompactum, P. chrysogenum, C. globosum, Candida albicans, Candida glabrata, Candida krusei, and Candida tropicalis.

In this method embodiment, illustratively, patient (e.g., human or animal) tissue can be received in 1) a 10% formalin fluid or 2) in a paraffin block in which the tissue has been fixed in formalin, such as 10% formalin. In one embodiment, the tissue can then be processed by various dehydration steps and finally embedded in paraffin. In this embodiment, the tissue can then be cut in 3 to 5 micron samples. In an illustrative embodiment, approximately 25 to 35 mg of tissue can then be processed as described in Examples 2 to 3 for mycotoxin extraction, using, for example, methanol for extraction. Illustratively, body fluids can be prepared as described in Examples 1 and 3 or by other methods known in the art. Illustratively, any antigen associated with a fungus or with a mycotoxin can be detected in this method embodiment. In another embodiment, any detection, identification, or quantitation method described in in U.S. Application Publication Number 2008/0014582, incorporated herein by reference, can be used.

In one illustrative embodiment, kits are provided. The kits are useful for identifying, detecting, or quantitating mycotoxins from a patient tissue or body fluid, or fungal DNA as described below. In one embodiment, the kit can contain one or more of the probes and/or primers described below, components to extract and isolate fungal DNA or mycotoxins, and/or components for DNA amplification, such as a heat stable DNA polymerase (e.g., Taq polymerase or Vent polymerase), buffers, MgCl₂, H₂O, and the like. In another embodiment, the kit can comprise any of the nucleic acids described herein. In one embodiment, the kit can contain components to extract (e.g., methanol) and/or isolate a mycotoxin described herein, such as antibody affinity matrices, ELISA plates, Luminex® beads, polyclonal or monoclonal antibodies, color development reagents, buffers, and the like. In another embodiment, the kit can contain negative and/or positive control samples and calibration reagents can be included in the kits. In one embodiment, the reagents can remain in liquid form. In another embodiment, the reagents can be lyophilized. In another illustrative embodiment, the kits can contain instructions for use.

In one embodiment, a calibration reagent (or multiple calibration reagents) can be included in the kit and “calibration reagent” for the purposes of any mycotoxin embodiment described in this patent application means any standard or reference material containing a known amount of the mycotoxin. In one aspect, the sample suspected of containing the mycotoxin and the calibration reagent (or multiple calibration reagents) are assayed under similar conditions, and the mycotoxin concentration is then calculated by comparing the results obtained for the unknown sample with the results obtained for the calibration reagent(s).

In one illustrative embodiment, the methods described above for mycotoxin detection, identification, or quantification can be combined with a method of identifying a specific fungal species in a patient tissue or a body fluid by identification of the DNA of the fungal species. The method of fungal DNA identification comprises extracting DNA of the fungal species from the patient tissue or the body fluid, amplifying the DNA, hybridizing a probe to the DNA to specifically identify the fungal species, and specifically identifying the fungal species. Thus, in one illustrative aspect, the method is based on both 1) amplification of fungal DNA using a PCR-based method and 2) detection, identification, and/or quantification of mycotoxins in patient body fluids and tissues. In one embodiment, the methods and compositions (e.g., primers and probes) for amplification of fungal DNA are highly specific and sensitive and avoid co-amplification of or do not co-amplify non-specific human or animal nucleic acids.

In some embodiments, real-time PCR-based methods can be used to amplify the fungal DNA and to detect and identify fungal DNA by hybridization of a probe to the fungal DNA. PCR is described in U.S. Pat. Nos. 4,683,202 and 4,800,159, incorporated herein by reference, and methods for PCR are well-known in the art. Real-time PCR combines amplification and simultaneous probe hybridization to achieve sensitive and specific detection of infectious molds (i.e., fungi) in real-time thereby providing instant detection and identification of molds. In this real-time PCR embodiment, the time to detect or identify the fungus and to obtain a diagnosis is greatly reduced. Real-time PCR is conducted according to methods well-known in the art. Exemplary probes and primers and their target DNAs, that can be used in combination with the methods for identifying, detecting, or quantitating mycotoxins as described herein are shown below. “Primer F” refers to a forward primer and “Primer R” refers to a reverse primer which are well-known terms in the art.

Target 1 - A. versicolor

| | |
|---|---|
| Probe 2 vers: | |
| 5′-cggggagccctctcgggggc | (SEQ ID NO: 1) |
| Primer F1: | |
| 5′-cgtaggtgaacctgcggaag | (SEQ ID NO: 2) |
| Primer R1: | |
| 5′-atcgatgccggaaccaagag | (SEQ ID NO: 3) |

Target 2 - A. niger

| | |
|---|---|
| Probe 3 niger: | |
| 5′-tgtctattgtacctgttgcttc | (SEQ ID NO: 4) |
| Primer F14: | |
| 5′-cgtaggtgaacctgcggaag | (SEQ ID NO: 5) |
| Primer R1: | |
| 5′-atcgatgccggaaccaagag | (SEQ ID NO: 6) |

Target 3 - P. chrysogenum

| | |
|---|---|
| Probe 4 chry: | |
| 5′-ctctgtctgaagattgtagtctgagt | (SEQ ID NO: 7) |
| Primer F1: | |
| 5′-cgtaggtgaacctgcggaag | (SEQ ID NO: 8) |
| Primer R1: | |
| 5′-atcgatgccggaaccaagag | (SEQ ID NO: 9) |

Target 4 - P. verrucosum

| | |
|---|---|
| Probe 5 verru: | |
| 5′-cccgcctttgctggccgcc | (SEQ ID NO: 10) |
| Primer F1: | |
| 5′-cgtaggtgaacctgcggaag | (SEQ ID NO: 11) |
| Primer R1: | |
| 5′-atcgatgccggaaccaagag | (SEQ ID NO: 12) |

Target 5 - A. flavus

| | |
|---|---|
| Probe 7 flav: | |
| 5′-cccgccattcatggccgccggg | (SEQ ID NO: 13) |
| Primer F1: | |
| 5′-cgtaggtgaacctgcggaag | (SEQ ID NO: 14) |
| Primer R1: | |
| 5′-atcgatgccggaaccaagag | (SEQ ID NO: 15) |

Target 6 - A. fumigatus

| | |
|---|---|
| Probe 8 fumi: | |
| 5′-aaagtatgcagtctgagttgattatc | (SEQ ID NO: 16) |
| Primer F1: | |
| 5′-cgtaggtgaacctgcggaag | (SEQ ID NO: 17) |
| Primer R1: | |
| 5′-atcgatgccggaaccaagag | (SEQ ID NO: 18) |

Target 7 - A. nidulans

| | |
|---|---|
| Probe 9 nid: | |
| 5′-cccagggggcgagccgccgg | (SEQ ID NO: 19) |
| Primer F1: | |
| 5′-cgtaggtgaacctgcggaag | (SEQ ID NO: 20) |
| Primer R1: | |
| 5′-atcgatgccggaaccaagag | (SEQ ID NO: 21) |

Target 8 - A. ochraceus

| | |
|---|---|
| Probe 10 ochr: | |
| 5′-acaccaacgtgaacactgtctgaag | (SEQ ID NO: 22) |
| Primer F1: | |
| 5′-cgtaggtgaacctgcggaag | (SEQ ID NO: 23) |
| Primer R1: | |
| 5′-atcgatgccggaaccaagag | (SEQ ID NO: 24) |

Target 9 - A. paraciticus

| | |
|---|---|
| Probe 11 para: | |
| 5′-cgggcccgccgtcatggccg | (SEQ ID NO: 25) |
| Primer F1: | |
| 5′-cgtaggtgaacctgcggaag | (SEQ ID NO: 26) |
| Primer R1: | |
| 5′-atcgatgccggaaccaagag | (SEQ ID NO: 27) |

Target 10 - A. sydowii

| | |
|---|---|
| Probe 12 syd: | |
| 5′-ccctcgggggcgagccgccg | (SEQ ID NO: 28) |
| Primer F1: | |
| 5′-cgtaggtgaacctgcggaag | (SEQ ID NO: 29) |
| Primer R1: | |
| 5′-atcgatgccggaaccaagag | (SEQ ID NO: 30) |

Target 11 - A. ustus

| | |
|---|---|
| Probe 13 ust: | |
| 5′-ccacaccgaacctcttgttatagc | (SEQ ID NO: 31) |
| Primer F1: | |
| 5′-cgtaggtgaacctgcggaag | (SEQ ID NO: 32) |
| Primer R1: | |
| 5′-atcgatgccggaaccaagag | (SEQ ID NO: 33) |

Target 12 - P. aurantiogriseum

| | |
|---|---|
| Probe 15 auran: | |
| 5′-cccgcctttactggccgccgg | (SEQ ID NO: 34) |
| Primer F1: | |
| 5′-cgtaggtgaacctgcggaag | (SEQ ID NO: 35) |
| Primer R1: | |
| 5′-atcgatgccggaaccaagag | (SEQ ID NO: 36) |

Target 13 - P. citrinum

| | |
|---|---|
| Probe 16 citr: | |
| 5′-tgttgcctcggcgggccccgc | (SEQ ID NO: 37) |
| Primer F4: | |
| 5′-ggaaggatcattaccgagtg | (SEQ ID NO: 38) |
| Primer R1: | |
| 5′-atcgatgccggaaccaagag | (SEQ ID NO: 39) |

Target 14 - P. corylophilum

| | |
|---|---|
| Probe 17 corylo: | |
| 5′-ttattgtaccttgttgcttcggcgg | (SEQ ID NO: 40) |
| Primer F1: | |
| 5′-cgtaggtgaacctgcggaag | (SEQ ID NO: 41) |
| Primer R1: | |
| 5′-atcgatgccggaaccaagag | (SEQ ID NO: 42) |

Target 15 - P. crustosum

| | |
|---|---|
| Probe 18 crust: | |
| 5′-cgatctccgggggacgggcc | (SEQ ID NO: 43) |
| Primer F7: | |
| 5′-ctgtccgagcgtcattgctg | (SEQ ID NO: 44) |
| Primer R5: | |
| 5′-cgaggaccggacgcggtg | (SEQ ID NO: 45) |

Target 16 - P. expansum

| | |
|---|---|
| Probe19expan: | |
| 5′-agacacccccgaactctgcctgaa | (SEQ ID NO: 46) |
| Primer F1: | |
| 5′-cgtaggtgaacctgcggaag | (SEQ ID NO: 47) |
| Primer R1: | |
| 5′-atcgatgccggaaccaagag | (SEQ ID NO: 48) |

Target 17 - P. fellutanum

| | |
|---|---|
| Probe 20 fell: | |
| 5′-cccgcctgccaggccgccg | (SEQ ID NO: 49) |
| Primer F1: | |
| 5′-cgtaggtgaacctgcggaag | (SEQ ID NO: 50) |
| Primer R1: | |
| 5′-atcgatgccggaaccaagag | (SEQ ID NO: 51) |

Target 18 - P. roquefortii

| | |
|---|---|
| Probe 21 rogue: | |
| 5′-cacccgtgtttatttaccttattgc | (SEQ ID NO: 52) |
| Primer F1: | |
| 5′-cgtaggtgaacctgcggaag | (SEQ ID NO: 53) |
| Primer R1: | |
| 5′-atcgatgccggaaccaagag | (SEQ ID NO: 54) |

Target 19 - P. simplicissimum

| | |
|---|---|
| Probe 22 simpl: | |
| 5′-cacccgtgtttatcgtaccttgttg | (SEQ ID NO: 55) |
| Primer F1: | |
| 5′-cgtaggtgaacctgcggaag | (SEQ ID NO: 56) |
| Primer R1: | |
| 5′-atcgatgccggaaccaagag | (SEQ ID NO: 57) |

Target 20: A. niger

| | |
|---|---|
| Probe: | |
| 5′-tgtctattgtaccctgttgcttc | (SEQ ID NO: 58) |
| Primer F: | |
| 5′-cgtaggtgaacctgcggaag | (SEQ ID NO: 59) |
| Primer R: | |
| 5′-atcgatgccggaaccaagag | (SEQ ID NO: 60) |

Target 21: A. terreus

| | |
|---|---|
| Probe: | |
| 5′-agtctgagtgtgattctttgcaatc | (SEQ ID NO: 61) |
| Primer F: | |
| 5′-acatgaaccctgttctgaaag | (SEQ ID NO: 62) |
| Primer R: | |
| 5′-ccaagagatccattgttgaaag | (SEQ ID NO: 63) |

Alternative illustrative embodiments for the Target 2 probe and primer F1 are 5′-cctctgccccccgggcccgtg (SEQ ID NO: 64) and 5′-ggaaggatcattaccgagtg (SEQ ID NO: 65), respectively. An alternative illustrative embodiment for the Target 7 probe is 5′-ggagccccccagggggcgag (SEQ ID NO: 66). An alternative illustrative embodiment for the Target 10 probe is 5′-cggggaaccccctcgggggc (SEQ ID NO: 67). An alternative illustrative embodiment for the Target 11 probe is 5′-tgcgctccccccgggggcag (SEQ ID NO: 68). Alternative illustrative embodiments for the Target 15 probe, primer F7, and primer R5 are 5′-ggccccgtcccccgatctccg (SEQ ID NO: 69), 5′-agtgaatcatcgagtctttgaac (SEQ ID NO: 70), and 5′-acctgatccgaggtcaacctg (SEQ ID NO: 71), respectively. An alternative illustrative embodiment for the Target 17 probe is 5′-cgggcccgcctgccaggccg (SEQ ID NO: 72). An alternative illustrative embodiment for the Target 18 probe is 5′-ccggggggtttacacccccg (SEQ ID NO: 73). An alternative illustrative embodiment for the Target 19 probe is 5′-ccggggggcatctgcccccgg (SEQ ID NO: 74).

Additional exemplary yeast probes and primers and their target DNAs, that can be used in combination with the methods for identifying, detecting, or quantitating mycotoxins as described herein are shown below. “P1” refers to the probe. “F1” refers to a forward primer and “R1” refers to a reverse primer which are well-known terms in the art.

| | | | | |
|---|---|---|---|---|
| Sequence | | | | |
| Descrip- | 5′ | | 3′ | Purifi- |
| tion | Mod | Sequence | Mod | cation |

Candida albicans

| | | | | |
|---|---|---|---|---|
| CA P1 | 6FAM | TCGGGGGCGGCCGCTGCGG | BHQ | Dual |
| (SEQ ID | | | #1 | HPLC |
| NO: 75) | | | | |
| CA F1 | | AAAAAGTACGTGAAATTGTTG | | Stnd. |
| (SEQ ID | | | | Desalt |
| NO: 76) | | | | |
| CA R1 | | AAGCCGTGCCACATTC | | Stnd. |
| (SEQ ID | | | | Desalt |
| NO: 77) | | | | |

Candida glabrata

| | | | | |
|---|---|---|---|---|
| CG P1 | 6FAM | ACCTAGGGAATGTGGCTCTGCG | BHQ | Dual |
| (SEQ ID | | | #1 | HPLC |
| NO: 78) | | | | |
| CG F1 | | TGGGCCAGCATCGGTTTTG | | Stnd. |
| (SEQ ID | | | | Desalt |
| NO: 79) | | | | |
| CG R1 | | CCTAGATAACAAGTATCGCAG | | Stnd. |
| (SEQ ID | | | | Desalt |
| NO: 80) | | | | |

Candida krusei

| | | | | |
|---|---|---|---|---|
| CK P1 | 6FAM | AAGGCGGTGTCCAAGTCCCTTG | BHQ | Dual |
| (SEQ ID | | | #1 | HPLC |
| NO: 81) | | | | |
| CK F1 | | TCAGTAGCGGCGAGTGAAG | | Stnd. |
| (SEQ ID | | | | Desalt |
| NO: 82) | | | | |
| CK R1 | | AGAAGGGCCTCACTGCTTC | | Stnd. |
| (SEQ ID | | | | Desalt |
| NO: 83) | | | | |

Candida tropicalis

| | | | | |
|---|---|---|---|---|
| CT P1 | 6FAM | TCGGGGGTGGCCTCTACAG | BHQ | Dual |
| (SEQ ID | | | #1 | HPLC |
| NO: 84) | | | | |
| CT F1 | | AAAAAGTACGTGAAATTGTTG | | Stnd. |
| (SEQ ID | | | | Desalt |
| NO: 85) | | | | |
| CT R1 | | AAGCCGTGCCACATTC | | Stnd. |
| (SEQ ID | | | | Desalt |
| NO: 86) | | | | |

In various embodiments, sample preparation (i.e., preparation of the target DNA) involves rupturing the cells (e.g., cells of the tissue or fungal spores in patient body fluid or tissue) and isolating the fungal DNA from the lysate. Techniques for rupturing cells and for isolation of DNA are well-known in the art. For example, cells may be ruptured by using a detergent or a solvent, such as phenol-chloroform, DNA may be separated from the lysate by physical methods including, but not limited to, centrifugation, pressure techniques, or by using a substance with affinity for DNA, such as, for example, silica beads, and after sufficient washing, the isolated DNA may be suspended in either water or a buffer. In other embodiments, commercial kits are available, such as Quiagen™, Nuclisensm™, and Wizard™ (Promega), and Promegam™. Methods for isolating DNA are described in Sambrook et al., “Molecular Cloning: A Laboratory Manual”, 3rd Edition, Cold Spring Harbor Laboratory Press, (2001), incorporated herein by reference.

In various embodiments described herein, the primers and probes used for amplification of the target DNA and for detection and identification of fungal DNA are oligonucleotides from about ten to about one hundred, more typically from about ten to about thirty or about six to about twenty-five base pairs long, but any suitable sequence length can be used. In illustrative embodiments, the primers and probes may be double-stranded or single-stranded, but the primers and probes are typically single-stranded. In one embodiment, the primers and probes described herein are capable of specific hybridization, under appropriate hybridization conditions (e.g., appropriate buffer, ionic strength, temperature, formamide, and MgCl₂ concentrations), to a region of the target DNA. In another embodiment, the primers and probes described herein are designed based on having a melting temperature within a certain range, and substantial complementarity to the target DNA. Methods for the design of primers and probes are described in Sambrook et al., “Molecular Cloning: A Laboratory Manual”, 3rd Edition, Cold Spring Harbor Laboratory Press, (2001), incorporated herein by reference.

In various illustrative embodiments, the primers and probes described herein for use in PCR can be modified by substitution, deletion, truncation, and/or can be fused with other nucleic acid molecules wherein the resulting primers and probes hybridize specifically to the intended targets and are useful in the methods described herein for amplification of the target DNAs. In other embodiments, derivatives can be made such as phosphorothioate, phosphotriester, phosphoramidate, and methylphosphonate derivatives, that specifically bind to single-stranded DNA or RNA (Goodchild, et al., Proc. Natl. Acad. Sci. 83:4143-4146 (1986)).

In one embodiment, the nucleic acids (i.e., the primers and probes) are isolated or substantially purified nucleic acids. A “purified” nucleic acid molecule is substantially free of other cellular material, or culture medium when produced by recombinant techniques, or substantially free of chemical precursors or other chemicals when chemically synthesized. An “isolated” nucleic acid is free of some sequences that naturally flank the nucleic acid in the genomic DNA of the organism from which the nucleic acid is derived. For example, in various embodiments, the isolated or purified nucleic acid molecule can contain less than about 5 kb, 4 kb, 3 kb, 2 kb, 1 kb, 0.5 kb, or 0.1 kb or can contain none of the nucleotide sequences that naturally flank the nucleic acid molecule in genomic DNA of the cell from which the nucleic acid is derived.

In other embodiments, nucleic acids complementary to the probes and primers described herein are contemplated, and those that hybridize to the nucleic acids described herein or those that hybridize to their complements under highly stringent conditions are contemplated for use in the methods described herein. “Highly stringent conditions” means hybridization at 65° C. in 5×SSPE and 50% formamide, and washing at 65° C. in 0.5×SSPE. Conditions for low stringency and moderately stringent hybridization are described in Sambrook et al., “Molecular Cloning: A Laboratory Manual”, 3rd Edition, Cold Spring Harbor Laboratory Press, (2001), incorporated herein by reference. In some illustrative aspects, hybridization may occur along the full-length of the nucleic acid.

In other embodiments, nucleic acid molecules can be used having about 60%, about 70%, about 75%, about 80%, about 85%, about 90%, about 95%, 96%, 97%, and 98% homology to the probes and primers described herein. Determination of percent identity or similarity between sequences can be done, for example, by using the GAP program (Genetics Computer Group, software; now available via Accelrys on http://www.accelrys.com), and alignments can be done using, for example, the ClustalW algorithm (VNTI software, InforMax Inc.). For example, a sequence database can be searched using the nucleic acid sequence of interest. In one aspect, algorithms for database searching are based on the BLAST software (Altschul et al., 1990). In some embodiments, the percent identity can be determined along the full-length of the nucleic acid.

As used herein, the term “complementary” refers to the ability of purine and pyrimidine nucleotide sequences to associate through hydrogen bonding to form double-stranded nucleic acid molecules. Guanine and cytosine, adenine and thymine, and adenine and uracil are complementary and can associate through hydrogen bonding resulting in the formation of double-stranded nucleic acid molecules when two nucleic acid molecules have “complementary” sequences. The complementary sequences can be DNA or RNA sequences. The complementary DNA or RNA sequences are referred to as a “complement.”

Techniques for synthesizing the probes and primers described herein are well-known in the art and include chemical syntheses and recombinant methods. Such techniques are described in Sambrook et al., “Molecular Cloning: A Laboratory Manual”, 3rd Edition, Cold Spring Harbor Laboratory Press, (2001), incorporated herein by reference. Primers and probes can also be made commercially (e.g., CytoMol, Sunnyvale, Calif. or Integrated DNA Technologies, Skokie, Ill.). Techniques for purifying or isolating the probes and primers described herein are well-known in the art. Such techniques are described in Sambrook et al., “Molecular Cloning: A Laboratory Manual”, 3rd Edition, Cold Spring Harbor Laboratory Press, (2001), incorporated herein by reference. The primers and probes described herein can be analyzed by techniques known in the art, such as restriction enzyme analysis or sequencing, to determine if the sequence of the primers and probes is correct.

In various embodiments of the methods and compositions described herein, the probes and primers can be labeled, such as with fluorescent compounds, radioactive isotopes, antigens, biotin-avidin, colorimetric compounds, or other labeling agents known to those of skill in the art, to allow detection and quantification of amplified DNA, such as by Real-Time PCR. In illustrative embodiments, the labels may include 6-carboxyfluorescein (FAM™), TET™ (tetrachloro-6-carboxyfluorescein), JOE™ (2,7,-dimethoxy-4,5-dichloro-6-carboxyfluorescein), VIC™, HEX (hexachloro-6-carboxyfluorescein), TAMRA™ (6-carboxy-N,N,N′,N′-tetramethylrhodamine), BHQ™, SYBR® Green, Alexa 350, Alexa 430, AMCA, BODIPY 630/650, BODIPY 650/665, BODIPY-FL, BODIPY-R6G, BODIPY-™ R, BODIPY-TRX, Cascade Blue, Cy3, Cy5,6-FAM, Fluorescein, Oregon Green 488, Oregon Green 500, Oregon Green 514, Pacific Blue, REG, Rhodamine Green, Rhodamine Red, ROX, and/or Texas Red.

Specificity of the probes and primers described herein was demonstrated by testing hybridization of the probe and primers sets against 23 different mold organisms (10 species of Aspergillus, 10 species of Penicillium, 2 species of Stachybotyrous, and 1 species of Fusarium). There were no cross-over reactions and no cross-over detection was noted for any of the tested probe and primer sequences. Thus, the primers and probes for amplification of fungal DNA are highly specific and avoid co-amplification of or do not co-amplify non-specific nucleic acids.

In one illustrative embodiment, universal probes can be used to provide a method for determining the presence of fungal DNA before conducting target-specific assays. In one embodiment, universal probes and primers can be used to detect the presence of Aspergillus and Penicillium species (see probes and primers for Fungal Universal Group 1 below). In this embodiment, the probes and primers can be homologous for all targets of interest related to Aspergillus and Penicillium species.

| Fungal Universal Group 1 | | |
|---|---|---|
| UP1: | 5′-cctcggatcaggtagggatac | (SEQ ID NO: 87) |
| UF1: | 5′-atgcctgtccgagcgtcatt | (SEQ ID NO: 88) |
| UR1: | 5′-ttcctccgcttattgatatg | (SEQ ID NO: 89) |

The following examples provide illustrative methods for carrying out the practice of the present invention. As such, these examples are provided for illustrative purposes only and are not intended to be limiting.

### EXAMPLE 1

### Samples and Sample Preparation

Human urine will be received in 5-10 ml quantities as first in the morning voided urines. Serums will be received with the blood clot removed prior to receipt and a minimum of 1 ml of serum will be frozen or used. Nasal secretions will be obtained from hospital patients or out-patients. Fixed autopsy and surgical biopsy specimens will be obtained from patients who had a history of exposure to mycotoxins or fungi. These samples will be obtained from hospital pathology departments or coroners' offices. Tissue samples and body fluid samples will also be obtained from patients who had no exposure to mycotoxins or fungi and will be sampled as a negative control group. Tissue specimens will be cut using procedures described in Example 2.

All specimens will be placed into two groups. Group 1 comprises samples from individuals with no reported symptoms or known fungi or mycotoxin exposure. These samples will serve as negative controls and n values will differ in each group of specimens. Group 2 comprises samples from individuals with reported exposure to non-identified fungi or chemicals. Each test conducted will have a different n value. Common symptoms of patients corresponding to group 2 samples may include blurred vision, memory loss, fatigue, headache, nausea, loss of balance, cognitive deficits, rhinitis, sinusitis, rashes, and allergies. A detailed history and symptoms will be provided to correspond to each patient sample.

Nasal secretions and washings will be obtained by injection of 3-5 ml of sterile saline in each nostril of a patient. The patient will be instructed to hold the saline in the nostrils for 30 seconds and then blow the saline into a sterile container held close to the nose. The specimen(s) will then be collected and placed in containers.

Negative control samples of mycotoxins will be made by dilution techniques for the mycotoxins described herein. Samples of extracted and filtered human heart tissue, liver tissue, urine, and nasal secretions (including sputum) will be spiked with various levels of the mycotoxins described herein. Each time a sample is evaluated, calibrators and negative and positive spiked tissues and fluids will also be evaluated. Statistical analysis on all types of samples for mycotoxins will be performed for sensitivity and specificity.

### EXAMPLE 2

### Preparation of Tissues for Mycotoxin Extraction

Preparation of tissues for myctotoxin extraction from formalin fixed tissue and paraffin-embedded tissue from humans or animals will be accomplished using the following procedure.

### Specimens

Tissue will be received as either tissue fixed in a 10% formalin solution or in a paraffin-embedded tissue block. Tissue can be stored indefinitely in either form. However, because of cross-linking of formalin and proteins which may give false negative readings for DNA, the tissue will not be stored in formalin for greater than 6 months. A minimum of 25-35 mg of formalin-fixed tissue will be required for mycotoxin extraction. A maximum of 3 grams of formalin-fixed tissue can be used.

### Materials

Phosphate Buffered Saline (PBS; 0.9%), acid-washed silica beads (Cat #G1277; obtained from Sigma-Aldrich), collection tubes (2 ml) screw cap, methanol (reagent grade, Sigma), and microcentrifuge tubes (2 ml) will be used.

### Procedure

For silica beads, 0.3 g±0.01 g of silica bead beating glass will be added to a 2 ml screw cap tube making sure that there are no glass beads in the cap or around the rim. The tubes containing the beads will be sterilized in an autoclave on the dry cycle for 10 minutes. If a large amount of tissue is evaluated, the tissue will be placed in a blender and blended in PBS until well emulsified in the PBS. The sample will then be filtered using simple gravity filtration through Whatman #9 filter paper.

The samples will be recorded and assigned numbers in a sample log. 25-35 mg of paraffin-embedded tissue will then be weighed and placed in a 2.0 ml screw cap tube. Methanol will be added (1.0 ml reagent grade methanol) to the tube with the 0.3 g of silica beads and the sample will be vortexed for 1 minute. The samples will be bead beated on the bead beater for 1 minute at the speed of 45. Then 500 μl of sample will be removed and placed in 4.5 ml of PBS taking care not to remove the paraffin from the sample tube. The sample could then be used for extraction or could be frozen at −20 degrees centigrade to be used later in extraction and detection of the mycotoxins described herein (see Example 3).

### EXAMPLE 3

### Preparation of Body Fluids for Mycotoxin Detection

Urine will be received from a morning fresh first-voided specimen and stored at 1-6 degrees centrigrade in a glass container. A urine analysis will be conducted using a dipstick to measure pH, specific gravity, glucose, nitrates, ketones, and blood. The urine will be examined for sediment and will be centrifuged at 2500 rpm for 5 minutes if sediment is present. The supernatant will be saved in a glass container for mycotoxin testing (storing in plastic will be avoided to avoid a decrease in the detection level of tricothecenes).

Nasal secretions and mucous samples as well as washes will be observed for mucous presence. If mucous is present, a solution of MUCOSOL™ (Alpha Tec Systems, Inc. Vancouver, Wash.) will be prepared and added in equal amounts of body fluid to MUCOSOL™ in the secretions containing mucous. The specimen will then be allowed to incubate 30 minutes at room temperature. The specimen will then be centrifuged and the supernatant will be removed. The sediment will then be re-suspended in 10 ml of PBS.

Blood samples will be obtained from the negative control group and exposed patients. Specimens will be allowed to clot (no anticoagulant added) and then centrifuged for 10 minutes at 2000 rpm. Specimens will be stored at 1-6 degrees centigrade for 48 hours or will be frozen at −20 degrees centigrade for an indefinite period of time. Blood samples will be extracted in a manner similar to that described by Garbis et al., Anal. Chem. 73:53589-64 (2001) and Hedman et al. Arch. Tieremahr. 50:13-24 (1997). Serum samples will be aliquoted in 200 μl amounts into sterile 1.5 ml polystyrene microcentrifuge tubes. Immediately, 600 μl of high performance HPLC grade acetonitrile (Fisher Scientific, Hampton, N.H.) will be added. After 15 minutes, the samples will be vortexed and centrifuged. The supernatants will be transferred into clean 1.5 ml glass vials. Each sample will be evaporated under a gentle stream of dry nitrogen and re-suspended in 100 μl of pre-warmed sterile water. This will be the final working solution for ELISA assays. Spinal fluid samples will be analyzed as obtained from human patients. Samples will not be processed before analysis.

### EXAMPLE 4

### Detection of Gliotoxin in Human Tissues and Human Body Fluids

Gliotoxin is a sulfur-containing mycotoxin produced by several species of fungi, including pathogens of humans such as Aspergillus fumigatus and also by species of Trichoderma, and Penicillium. The methods described were validated as a semiquantitative test and reported out “Positive”, “Negative”, or “Equivocal”. Values were also reported as ng/dl (ppb). Values were determined and reported in parts per billion (ppb). The test was a Laboratory Determined Test (LDT) and validated at RTL in Carrollton, Tex. using an ELISA plate with reagents to determine the levels of Gliotoxin in human body fluids and tissues. The test used bis(methylthio)gliotoxin (SS′-dimethyl-gliotoxin (bmGT)) as a diagnostic marker of pathologies caused by gliotoxin-producing fungi or their derivatives. bmGT is a metabolite and an analog of gliotoxin (GT) shown to be a more sensitive marker than GT in the diagnosis of aspergillosis. Results have shown that bmGT can be detected in biological samples of immunodepressed patients with a high reliability, sensitivity and specificity. All ELISA tests were validated using Analyte Specific Reagents (ASRs) from Beacon Analytical Systems, Inc. (Saco, Me.). Structures of gliotoxin and Bis-gliotoxin are shown in FIG. 1: Panel A shows Gliotoxin (GT) and Panel B shows bis(methylthio)gliotoxin (SS′-dimethyl-gliotoxin—(bmGT-).

### Competitive Direct Enzyme-Linked Immunosorbent Assay (ELISA)

A competitive direct enzyme-linked immunosorbent assay (ELISA) was performed, which allows detection of concentrations in parts per billion (ppb). Gliotoxin antigens in the patient samples and controls compete with enzyme-labeled bmGT-HRP (conjugate) for the antibody binding sites inside the surface of the testing wells. After a wash step, substrate was added that reacted with the bound conjugate to produce a blue color. Addition of stop solution halted the reaction and changes the color to yellow.
Darker color=Lower concentration
Lighter color=Higher concentration

The test was read in a microwell reader to yield optical densities. The optical densities of the controls formed the standard curve. The sample optical densities were plotted against the curve to calculate the exact concentration of bis(methylthio)gliotoxin is the samples.
Specimens: Urine specimens were collected in a supplied RTL plastic tube (plastic is preferred because of safety issues) and stored at 2-6° C. If specimen is to be held more than one week, specimens can be frozen at −10 to −25.9° C. All urine specimens were diluted 1:5 in 10% MeOH/PBS for testing. After testing, all specimens were frozen in a −10 to −26° C. freezer and kept for a minimum of 6 months prior to disposal.

Serum specimens were collected in a serum separator tube, centrifuged, and stored at 2-6° C. If specimen is to be held more than one week, serum specimens can be frozen at −10 to −25.9° C. After testing, all specimens were aliquoted to a new storage tube and frozen in a −10 to −26° C. freezer and kept for a minimum of 6 months prior to disposal.
Materials: bmGT Test using the Bis MethylthioGliotoxin (bmGT) 96 antibody-coated microwells (ELISA wells) (Beacon Analytical Systems Inc, Saco, Me.); Bis MethylthioGliotoxin (bmGT) ELISA Kit—96 antibody-coated microwells (ELISA wells) (Beacon Analytical Systems Inc, Saco, Me.); Bis MethylthioGliotoxin (bmGT) ELISA Kit with 5 green capped brown bottles of 0, 0.3, 1, 3, and 10 ppb calibrators (Beacon Analytical Systems Inc, Saco, Me.); Bis MethylthioGliotoxin (bmGT) ELISA Kit—HRP conjugate solution diluent (Beacon Analytical Systems Inc, Saco, Me.); Bis MethylthioGliotoxin (bmGT) ELISA Kit—HRP conjugate. Dilute 1:1500 using provided diluent solution prior to use (Beacon Analytical Systems Inc, Saco, Me.); Bis MethylthioGliotoxin (bmGT) ELISA Kit Substrate Solution (Beacon Analytical Systems Inc, Saco, Me.); Bis MethylthioGliotoxin (bmGT) ELISA Kit—clear Stop Solution (Beacon Analytical Systems Inc, Saco, Me.); Bis MethylthioGliotoxin (bmGT) ELISA Kit Wash solution (Beacon Analytical Systems Inc, Saco, Me.); bmGT High, Low and Negative Controls (Created in house from purchased stocks; 10% MeOH/PBS (Various Vendors); Mucosol (Various Vendors); Molecular grade water (Various Vendors). Calculations for determinations were made knowing the exposure time of the substrate to the antigen/antibody mixture.
Quality Control: Samples were validated for the semiquantitative determination of bmGT. Five bmGT calibrators were processed along with the patient samples. The calibrators were provided at 0, 0.3, 1, 3, and 10 ppb and during analysis a semi-log curve fit for the standard curve was used to plot the points of the calibrators. A correlation coefficient of >95% was acceptable. Three bmGT controls were created by RTL and processed along with the patient samples and calibrators. These three controls included a high bmGT control, a low bmGT control, and a negative bmGT control. Calculations embedded in UNIFlow were used to perform analysis of Gliotoxin testing. Data was analyzed using UNIFlow software.
Sample Preparation: Controls were made in a negative urine sample (<0.25 ppb gliotoxin). Urine samples were diluted 1:5 in a 10% MeOH/PBS solution to remove the matrix effect of the urine.
Procedure: 100.0 μl of calibrators, controls, and samples to bmGT antibody-coated wells. The standards and samples were added in ascending order, and the controls were added as high, low, and negative in order. After pipetting into the wells, the tray was placed on a shaker at 80-100 rpm for 15 minutes and allowed to incubate. After incubation, 100.0 μL of conjugate (prepared by adding 8.0 μL of Bis-gliotoxin Enzyme Conjugate to 12 mL of HRP-gliotoxin Diluent) was added to all wells. After pipetting into the wells, the tray was placed on a shaker at 80-100 rpm for 15 minutes and allowed to incubate. Wells were washed 5 times with Beacon Wash Solution. Any residual solution was wiped on the outside of the wells with the paper towel. 100.0 μl of substrate was added to all the wells. The tray was placed on the shaker for 30 minutes to incubate. 100.0 μl of stop solution was added and the tray was placed back on shaker for 5 minutes, and the plate was read using a SpectraMax 190 Microplate Reader and SoftMax Pro 4.8 software. Once the run was complete and accepted all samples were stored.
Data Analysis: The UNIFlow statistics software was used to plot the calibrators into a semi-log curve to generate a standard curve. Controls and samples were plotted on a graph to give results in parts per billion (ppb) or nanograms/ml.

### Results:

Five bmGT calibrators were provided and processed along with the patient samples. The calibrators were provided at 0, 0.3, 1, 3, and10 ppb and during analysis a semi-log curve fit for the standard curve was used to plot the points of the calibrators. A correlation coefficient of >95% was acceptable.

Three bmGT controls were created by RTL and processed along with the patient samples and calibrators. These three controls included a high bmGT control, a low bmGT control and a bmGT control. To determine if these controls were acceptable and in range they are compared to the current control ranges which are provided to the lab and recalculated and updated with each lot of control. For run acceptance two of the three controls must be within the current control ranges, and the negative control must not be “Equivocal” or “Positive.”

If the above calibrators and controls were approved then results were determined to be “Positive” or “Negative”, or “Equivocal” based on the standard curve analysis. Limit of Detection in this test was determined to be 0.25 ppb. Thus any values less than 0.25 ppb were reported as “Negative”. Values of 0.25 or greater were reported as “Positive”. Values of 0.20-0.24 were reported as “Equivocal”. If the processed sample results, before the factoring dilution, were greater than the highest calibration sample (10.0 ppb), the sample was reported as “greater than AMR (Analytical Measurement Range)”. Results are shown below and in FIGS. 2, 3, 4, and 5.

TABLE 1
Test Samples*

| | Sample ID | | Abs. | logit B/Bo | ppb |
|---|---|---|---|---|---|
| 1 | control 1 | ee | 1.089 | 1.070 | 0.219 |
| 2 | control 2 | g | 1.104 | 1.153 | 0.173 |
| 3 | control 3 | q | 1.093 | 1.091 | 0.207 |
| 4 | control 4 | pp | 1.165 | 1.848 | 0.023 |
| 5 | 115 | 143115 | 0.867 | 0.440 | 1.356 |
| 6 | 128 | 143128 | 0.297 | −0.473 | 18.990 |
| 7 | 104 | 143104 | 0.827 | 0.367 | 1.672 |
| 8 | 95 | 14095 | 0.620 | 0.043 | 4.271 |

*samples 5 to 8 = undiluted test samples

### Sample Summary

Controls (4 samples): 0.12 ppb-1.1 ppb
143115—Symptoms: sinus, skin problems
   Aflatoxin (−) Ochratoxin (−) Trichothecenes (−) Gliotoxin levels: 1.36ppb
143104—Symptoms: Allergy, ear, mouth/throat, neurological, sinus problems
   Aflatoxin (−) Ochratoxin (+, 8.5ppb)Trichothecenes (−) Gliotoxin levels: 1.68 ppb
143095—Symptoms: high allergy, sinus, joint and weight problems
   Aflatoxin (−) Ochratoxin (−) Trichothecenes (+, 2.56 ppb) Gliotoxin levels: 4.3 ppb
143128—Symptoms: ear problems
   Aflatoxin (−) Ochratoxin (+, 13.4 ppb)Trichothecenes (+, 3ppb) Gliotoxin levels: 19.0 ppb

### EXAMPLE 5

### Detection of Gliotoxin in Human Tissues and Human Body Fluids

Assays similar to those shown in Example 4 were performed using Chaetoglobosin A. Results for the standard curve and negative controls for Chaetoglobosin A are shown in FIG. 6. Structures are shown for Chaetoglobosum (FIG. 7) and Chaetoglobosin A and Chaetoglobosin C (FIG. 8, Panels A and B, respectively).

### EXAMPLE 6

### Mycophelonic Acid (MPA) Determination

Assays similar to those shown in Example 4 were performed using Mycophelonic Acid (MPA). Briefly, a competitive enzyme labeled immunoassay was performed. The residues were extracted from samples by mixing with 10% methanol/PBS Buffer (pH 7.1). The extracts were tested in the immunoassay. MPA-HRP enzyme conjugate was pipetted into the test wells followed by calibrators or sample extracts. MPA antibody was pipetted into the test wells to initiate the reaction. During the 30 minute incubation period, MPA residues compete for binding to MPA antibody which in turn, binds to the test well. Following the 30 minute incubation, the contents of the well were removed and the wells washed to remove any unbound toxin or enzyme-labeled toxin. A clear substrate was then added to the wells and any bound enzyme-toxin conjugate caused the conversion to a blue color during a 30 minute incubation period. The reaction was stopped and amount of color in each well was read using a SpectraMax 190 Microplate Reader. The color of unknown samples was compared to the color of the calibrators, and the MPA concentrations of the samples were determined.

Reagents and samples (urine or environmental samples) were allowed to reach room temperature prior to running the test. The test wells were placed in the plate. 50 ul of Enzyme Conjugate was added to each test well. 50 ul of calibrators and/or samples were added to the appropriate test wells. 50 ul of Antibody Solution was added to each test well. Test wells were shaken and incubated for 30 minutes. The contents of each well were discarded and each well filled with distilled or deionized water. Wells were inverted onto absorbent paper to remove last of wash solution. 100 ul of Substrate was dispensed into each well. Plates were shaken and incubated for 30 minutes. 100 ul of Stop solution was dispensed into each test well and shaken gently to mix.

TABLE 2
Calculations for MPA:

| Concentration | | OD (450 nm) | Mean OD | % Bo ** |
|---|---|---|---|---|
| 0 | ppb | 1.960/1.901 | 1.931 | 100% |
| 0.3 | ppb | 1.772/1.665 | 1.718 | 89% |
| 3.0 | ppb | 1.161/1.209 | 1.185 | 61% |
| 30.0 | ppb | 0.440/0.452 | .0446 | 23% |

** % Bo equals average sample absorbance divided by average negative control absorbance multiplied by 100%.

1. A method of identifying a gliotoxin, or a derivative thereof, a mycotoxin of a Penicillium species, or a mycotoxin of a Chaetomium species in a patient tissue or a body fluid, the method comprising:
extracting the mycotoxin from the patient tissue or the body fluid;
contacting the mycotoxin with an antibody directed against the mycotoxin; and
identifying the mycotoxin wherein the mycotoxin is a gliotoxin, or a derivative thereof, a mycotoxin of a Penicillium species, or a mycotoxin of a Chaetomium species.

2. The method of claim 1 further comprising quantifying the mycotoxin.

3. The method of claim 1 wherein the body fluid is selected from the group consisting of urine, nasal secretions, nasal washes, bronchial lavages, bronchial washes, spinal fluid, sputum, gastric secretions, seminal fluid, other reproductive tract secretions, lymph fluid, whole blood, serum, and plasma.

4. The method of claim 1 wherein the mycotoxin is a gliotoxin derivative.

5. The method of claim 4 wherein the gliotoxin derivative is Bis-(methylthio)gliotoxin.

6. The method of claim 1 wherein the mycotoxin is a mycotoxin of a Penicillium species.

7. The method of claim 6 wherein the mycotoxin is mycophenolic acid.

8. The method of claim 1 wherein the mycotoxin is a mycotoxin of a Chaetomium species.

9. The method of claim 8 wherein the mycotoxin is selected from the group consisting of emodins, chrysophanols, chaetoglobosins A, B, C, D, E and F, chetomins, azaphilones, and chaetoviridins.

10. The method of claim 9 wherein the mycotoxin is chaetoglobosin A or B.

11. The method of claim 1 wherein the antibody is a polyclonal antibody.

12. The method of claim 1 wherein the antibody is a monoclonal antibody.

13. The method of claim 2 wherein the sensitivity of the quantitation is at least 0.2 ng/ml.

14. The method of claim 5 wherein there is no other mycotoxin detected.

15. The method of claim 7 wherein there is no other mycotoxin detected.

16. The method of claim 10 wherein there is no other mycotoxin detected.

17. The method of claim 1 wherein the mycotoxin is contacted with the antibody using an enzyme-linked immunosorbent assay.

18. The method of claim 1 further comprising identifying the mycotoxin using negative and positive control samples.

19. The method of claim 1 further comprising using calibration reagents to quantify the mycotoxin.

20. The method of claim 1 wherein methanol is used for the extraction.

21.-112. (canceled)
